(19) United States
(12) Patent Application Publication
(10) Pub. No.: US 2007/0129629 A1
(43) Pub. Date: Jun. 7, 2007

(54) System and method for surgical navigation

(76) Inventors: Gerald Lee Beauregard, Stratham, NH (US); Daniel Eduardo Groszmann, Cambridge, MA (US); Peter Traneus Anderson, Andover, MA (US); Ragu Sampathkumar, Somerville, MA (US); Jonathan David Schiff, Andover, MA (US)

Correspondence Address: MCANDREWS HELD & MALLOY, LTD, 500 WEST MADISON STREET, SUITE 3400, CHICAGO, IL, 60661, US

(21) Appl. No.: 11/286,777

(22) Filed: Nov. 23, 2005

(51) Int. Cl. G01C 21/00; A61B 5/05

(52) U.S. Cl. 600/424; 701/207

(57) ABSTRACT

Certain embodiments of the present invention provide an integrated medical navigation system for use with an electromagnetic sensor and a device that may comprise a navigation interface configured to receive digitized signals from an electromagnetic sensor, a tracker module configured to determine a location of a device based on the received digitized signals; and a navigation module configured to receive the location determined by the tracking module, and register the location to acquired patient image data.

### RELATED APPLICATIONS [Not Applicable]

### FEDERALLY SPONSERED RESEARCH OR DEVELOPMENT [Not Applicable]

### MICROFICHE/COPYRIGHT REFERENCE [Not Applicable]

### BACKGROUND OF THE INVENTION

The present invention generally relates to image-guided surgery (or surgical navigation). In particular, the present invention relates to a medical navigation system with a reduced footprint that improves operating room ergonomics.

Medical navigation systems track the precise location of surgical instruments in relation to multidimensional images of a patient's anatomy. Additionally, medical navigation systems use visualizations tools to provide the surgeon with co-registered views of these surgical instruments with the patient's anatomy. This functionality is typically provided by including components of the medical navigation system on a wheeled cart (or carts) that can be moved throughout the operating room. However, it would be desirable to provide a medical navigation system with a reduced footprint to improve operating room ergonomics and enable new applications for surgical navigation technology.

### BRIEF SUMMARY OF THE INVENTION

Certain embodiments of the present invention provide an integrated medical navigation system for use with an electromagnetic sensor and a device comprising a navigation interface configured to receive digitized signals from an electromagnetic sensor, a tracker module configured to determine a location of a device based on the received digitized signals, and a navigation module configured to receive the location determined by the tracking module, and register the location to acquired patient image data.

Certain embodiments of the present invention provide a portable medical navigation system for use with an electromagnetic sensor and a device comprising a portable computer having a small footprint, a navigation interface housed in the portable computer and configured to receive digitized signals from an electromagnetic sensor, a tracker module configured to determine a location of a device based on the received digitized signals, and a navigation module configured to receive the location determined by the tracker module, and register the location to acquired patient image data.

Certain embodiments of the present invention provide a method for operating a medical navigation system with an electromagnetic sensor and a device, the method comprising receiving digitized signals from an electromagnetic sensor through an interface, determining a location of a device based on the received digitized signals; and registering the location to acquired patient image data.

Certain embodiments of the present invention provide a portable medical navigation system for use with an electromagnetic sensor and a device comprising a portable computer having a small footprint, a navigation interface housed in the portable computer and configured to receive digitized signals from an electromagnetic sensor, a first processor housed in the portable computer and configured to determine a location of a device based on the received digitized signals, and a second processor housed in the portable computer and configured to receive the location determined by the first processor over a local interface, and register the location to acquired patient image data.

### BRIEF DESCRIPTION OF SEVERAL VIEWS OF THE DRAWINGS

FIG. 1 illustrates a medical navigation system used in accordance with an embodiment of the present invention.

FIG. 2 illustrates a medical navigation system used in accordance with an embodiment of the present invention.

FIG. 3 illustrates a medical navigation system used in accordance with an embodiment of the present invention.

### DETAILED DESCRIPTION OF THE INVENTION

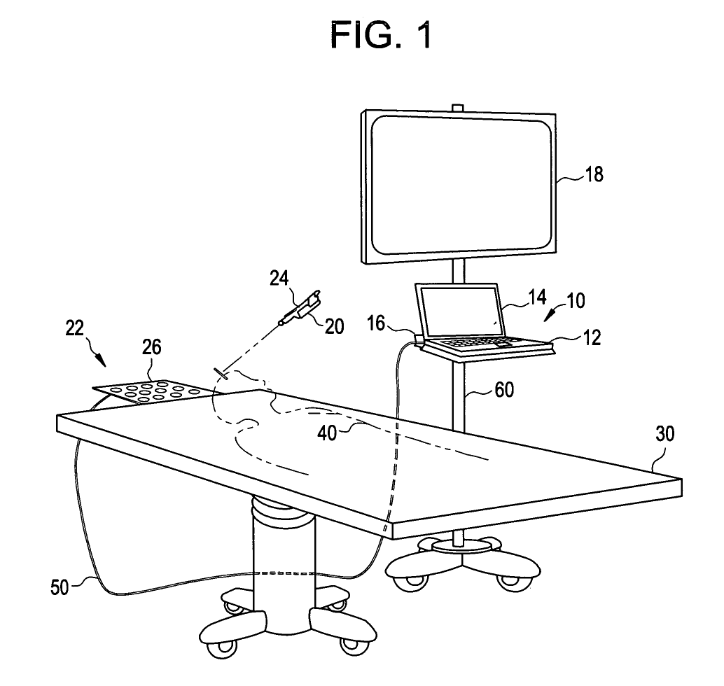

Referring now to FIG. 1, a medical navigation system (e.g., a surgical navigation system), designated generally by reference numeral 10, is illustrated as including a portable computer 12, a display 14, and a navigation interface 16. The medical navigation system 10 is configured to operate with an electromagnetic field generator 20 and electromagnetic sensor 22 to determine the location of a device 24.

A table 30 is positioned near the electromagnetic sensor 22 to support a patient 40 during a surgical procedure. A cable 50 is provided for the transmission of data between, the electromagnetic sensor 22 and the medical navigation system 10. The medical navigation system 10 is mounted on a portable cart 60 with a second display 18 in the embodiment illustrated in FIG. 1.

The electromagnetic sensor 22 may be a printed circuit board. Certain embodiments may include an electromagnetic sensor 22 comprising a printed circuit board receiver array 26 including a plurality of coils and coil pairs and electronics for digitizing magnetic field measurements detected in the printed circuit board receiver array 26. The magnetic field measurements can be used to calculate the position and orientation of the electromagnetic field generator 20 according to any suitable method or system. After the magnetic field measurements are digitized using electronics on the electromagnetic sensor 22, the digitized signals are transmitted to the navigation interface 16 through cable 50. As will be explained below in detail, the medical navigation system 10 is configured to calculate a location of the device 24 based on the received digitized signals.

The medical navigation system 10 described herein is capable of tracking many different types of devices during different procedures. Depending on the procedure, the device 24 may be a surgical instrument (e.g., an imaging catheter, a diagnostic catheter, a therapeutic catheter, a guidewire, a debrider, an aspirator, a handle, a guide, etc.), a surgical implant (e.g., an artificial disk, a bone screw, a shunt, a pedicle screw, a plate, an intramedullary rod, etc.), or some other device. Depending on the context of the usage of the medical navigation system 10, any number of suitable devices may be used.

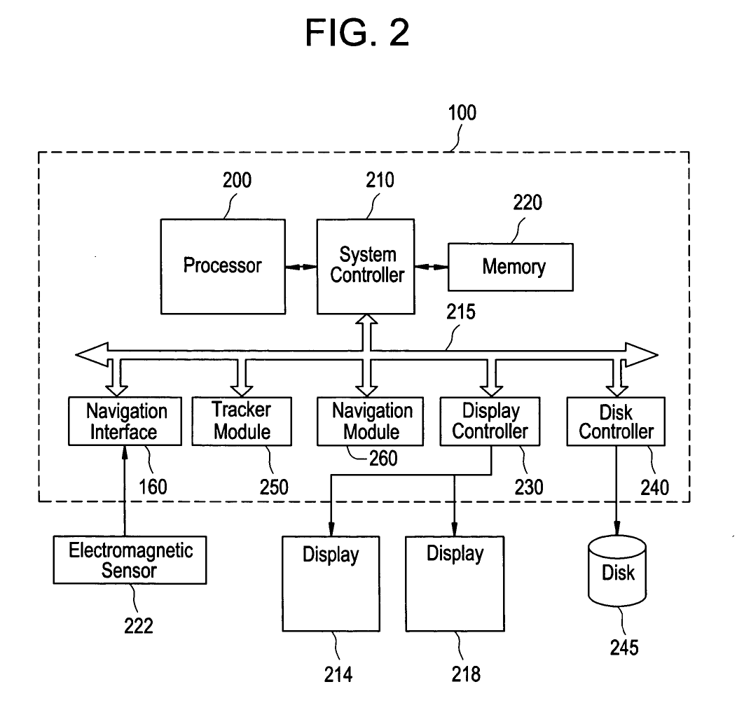

With regards to FIG. 2, an exemplary block diagram of the medical navigation system 100 is provided. The medical navigation system 100 is illustrated conceptually as a collection of modules, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as a dedicated processor for visualization operations. As a further option, the modules may be implemented using a hybrid configuration in which certain modular functions are performed using dedicated hardware, while the remaining modular functions are performed using an off-the-shelf computer. The operations of the modules may be controlled by a system controller 210.

The navigation interface 160 receives digitized signals from an electromagnetic sensor 222. In the embodiment illustrated in FIG. 1, the navigation interface 16 includes an Ethernet port. This port may be provided, for example, with an Ethernet network interface card or adapter. However, according to various alternate embodiments, the digitized signals may be transmitted from the electromagnetic sensor 222 to the navigation interface 160 using alternative wired or wireless communication protocols and interfaces.

The digitized signals received by the navigation interface 160 represent magnetic field information detected by an electromagnetic sensor 222. In the embodiment illustrated in FIG. 2, the navigation interface 160 transmits the digitized signals to the tracker module 250 over a local interface 215. The tracker module 250 calculates position and orientation information based on the received digitized signals. This position and orientation information provides a location of a device.

The tracker module 250 communicates the position and orientation information to the navigation module 260 over a local interface 215. As an example, this local interface 215 is a Peripheral Component Interconnect (PCI) bus. However, according to various alternate embodiments, equivalent bus technologies may be substituted without departing from the scope of the invention.

Upon receiving the position and orientation information, the navigation module 260 is used to register the location of the device to acquired patient data. In the embodiment illustrated in FIG. 2, the acquired patient data is stored on a disk 245. The acquired patient data may include computed tomography data, magnetic resonance data, positron emission tomography data, ultrasound data, X-ray data, or any other suitable data, as well as any combinations thereof. By way of example only, the disk 245 is a hard disk drive, but other suitable storage devices and/or memory may be used.

The acquired patient data is loaded into memory 220 from the disk 245. The navigation module 260 reads from memory 220 the acquired patient data. The navigation module 260 registers the location of the device to acquired patient data, and generates image data suitable to visualize the patient image data and a representation of the device. In the embodiment illustrated in FIG. 2, the image data is transmitted to a display controller 230 over a local interface 215. The display controller 230 is used to output the image data to two displays 214 and 218.

While two displays 214 and 218 are illustrated in the embodiment in FIG. 2, alternate embodiments may include various display configurations. Various display configurations may be used to improve operating room ergonomics, display different views, or display information to personnel at various locations. For example, as illustrated in FIG. 1, a first display 14 may be included on the medical navigation system 10, and a second display 18 that is larger than first display 14 is mounted on a portable cart 60. Alternatively, one or more of the displays 214 and 218 may be mounted on a surgical boom. The surgical boom may be ceiling-mounted, attachable to a surgical table, or mounted on a portable cart.

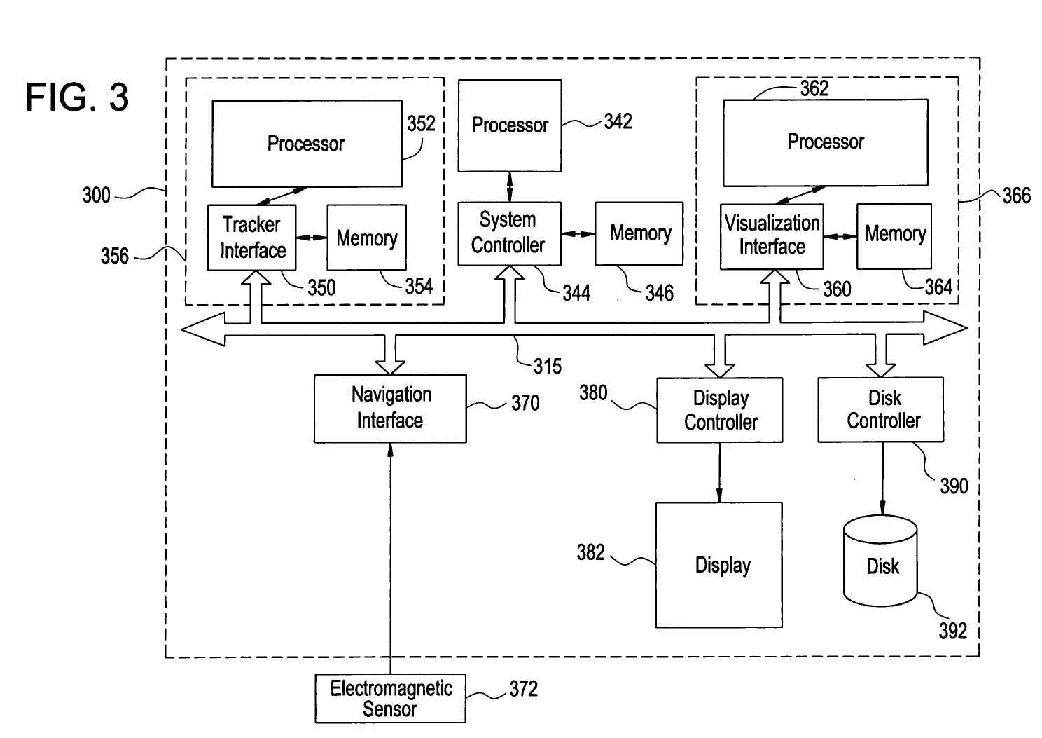

Referring now to FIG. 3, an alternative embodiment of a medical navigation system 300 is illustrated. The medical navigation system 300 comprises a portable computer with a relatively small footprint (e.g., approximately 1000 cm²) and an integrated display 382. According to various alternate embodiments, any suitable smaller or larger footprint may be used.

The navigation interface 370 receives digitized signals from an electromagnetic sensor 372. In the embodiment illustrated in FIG. 3, the navigation interface 370 transmits the digitized signals to the tracker interface 350 over a local interface 315. In addition to the tracker interface 350, the tracker module 356 includes a processor 352 and memory 354 to calculate position and orientation information based on the received digitized signals.

The tracker interface 350 communicates the calculated position and orientation information to the visualization interface 360 over a local interface 315. In addition to the visualization interface 360, the navigation module 366 includes a processor 362 and memory 364 to register the location of the device to acquired patient data stored on a disk 392, and generates image data suitable to visualize the patient image data and a representation of the device.

The visualization interface 360 transmits the image data to a display controller 380 over a local interface 315. The display controller 380 is used to output the image data to display 382.

The medical navigation system 300 also includes a processor 342, system controller 344, and memory 346 that are used for additional computing applications such as scheduling, updating patient data, or other suitable applications. Performance of the medical navigation system 300 is improved by using a processor 342 for general computing applications, a processor 352 for position and orientation calculations, and a processor 362 dedicated to visualization operations. Notwithstanding the description of the embodiment of FIG. 3, alternative system architectures may be substituted without departing from the scope of the invention.

Several embodiments are described above with reference to drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any machine-readable media for accomplishing its operations. As noted above, the embodiments of the present invention may be implemented using an existing computer processor, or by a special purpose computer processor incorporated for this or another purpose or by a hardwired system.

As noted above, embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments of the invention are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

Those skilled in the art will appreciate that the embodiments disclosed herein may be applied to the formation of any medical navigation system. Certain features of the embodiments of the claimed subject matter have been illustrated as described herein, however, many modifications, substitutions, changes and equivalents will now occur to those skilled in the art. Additionally, while several functional blocks and relations between them have been described in detail, it is contemplated by those of skill in the art that several of the operations may be performed without the use of the others, or additional functions or relationships between functions may be established and still be in accordance with the claimed subject matter. It is, therefore, to be understood that the appended claims are intended to cover all such modifications and changes as fall within the true spirit of the embodiments of the claimed subject matter.

1. An integrated medical navigation system for use with an electromagnetic sensor and a device comprising:
a navigation interface configured to receive digitized signals from an electromagnetic sensor;
a tracker module configured to determine a location of a device based on the received digitized signals; and
a navigation module configured to receive the location determined by the tracking module, and register the location to acquired patient image data.

2. The medical navigation system of claim 1, wherein the navigation interface is a wired interface.

3. The medical navigation system of claim 2, wherein the wired interface is an Ethernet port.

4. The medical navigation system of claim 1, wherein the navigation interface is a wireless interface.

5. The medical navigation system of claim 4, wherein the wireless interface is an IEEE 802.11 compatible interface.

6. The medical navigation system of claim 1, wherein the acquired patient image data is selected from the group consisting of computed tomography data, magnetic resonance data, positron emission tomography data, ultrasound data, and X-ray data and any combinations thereof.

7. A portable medical navigation system for use with an electromagnetic sensor and a device comprising:
a portable computer having a small footprint;
a navigation interface housed in the portable computer and configured to receive digitized signals from an electromagnetic sensor;
a tracker module configured to determine a location of a device based on the received digitized signals; and
a navigation module configured to receive the location determined by the tracker module, and register the location to acquired patient image data.

8. The medical navigation system of claim 7, further comprising a display to visualize the patient image data and a representation of the device.

9. The medical navigation system of claim 8, wherein the display is housed in the portable computer.

10. The medical navigation system of claim 9, further comprising a second display to visualize the patient image data and a representation of the device.

11. The medical navigation system of claim 8, wherein the display is mounted on a surgical boom.

12. The medical navigation system of claim 8, wherein the display is mounted on a portable cart.

13. The medical navigation system of claim 8, wherein the navigation interface is a wired interface.

14. The medical navigation system of claim 13, wherein the wired interface is an Ethernet port.

15. The medical navigation system of claim 8, wherein the navigation interface is a wireless interface.

16. The medical navigation system of claim 15, wherein the wireless interface is an IEEE 802.11 compatible interface.

17. The medical navigation system of claim 8, wherein the acquired patient image data is selected from the group consisting of computed tomography data, magnetic resonance data, positron emission tomography data, ultrasound data, and X-ray data and any combinations thereof.

19. The medical navigation system of claim 7, wherein the device comprises a surgical instrument selected from the group consisting of a catheter, a guidewire, a debrider, an aspirator, and any combinations thereof.

20. The medical navigation system of claim 7, wherein the device comprises a surgical implant.

21. The medical navigation system of claim 20, wherein the surgical implant is selected from the group consisting of an artificial disk, a bone screw, a shunt, a pedicle screw, a plate, and any combinations thereof.

22. A method for operating a medical navigation system with an electromagnetic sensor and a device, the method comprising:
receiving digitized signals from an electromagnetic sensor through an interface;
determining a location of a device based on the received digitized signals; and
registering the location to acquired patient image data.

23. A machine-readable storage medium holding code for performing the method according to claim 22.

24. A portable medical navigation system for use with an electromagnetic sensor and a device comprising:
a portable computer having a small footprint;
a navigation interface housed in the portable computer and configured to receive digitized signals from an electromagnetic sensor;
a first processor housed in the portable computer and configured to determine a location of a device based on the received digitized signals; and
a second processor housed in the portable computer and configured to receive the location determined by the first processor over a local interface, and register the location to acquired patient image data.

25. The medical navigation system of claim 24, wherein the local interface is a PCI bus.

26. The medical navigation system of claim 24, wherein the local interface is a PCI Express bus.

27. The medical navigation system of claim 24, further comprising a display to visualize the patient image data and a representation of the device.

28. The medical navigation system of claim 27, wherein the display is housed in the portable computer.

29. The medical navigation system of claim 28, wherein the portable computer is mounted on a portable cart.

30. The medical navigation system of claim 24, wherein the navigation interface is a wired interface.

31. The medical navigation system of claim 24, wherein the navigation interface is a wireless interface.
